(19) United States
(12) Patent Application Publication
(10) Pub. No.: US 2026/0138957 A1
(43) Pub. Date: May 21, 2026

(54) UREA-LINKED 2-AMINOIMIDAZOLE DIMER POTENTIATORS

(71) Applicant: UNIVERSITY OF NOTRE DAME DU LAC, South Bend, IN (US)

(72) Inventors: Christian MELANDER, South Bend, IN (US); Veronica B. HUBBLE, South Bend, IN (US); Santiana MARRUJO, South Bend, IN (US)

(73) Assignee: UNIVERSITY OF NOTRE DAME DU LAC, South Bend, IN (US)

(21) Appl. No.: 19/123,404

(22) PCT Filed: Oct. 26, 2023
(86) PCT No.: PCT/US2023/077945

§ 371 Date: Apr. 23, 2025

Related U.S. Application Data
(60) Provisional application No. 63/380,957, filed on Oct. 26, 2022.

(51) Int. Cl. C07D 233/88; A61K 31/4178; A61K 31/7048; A61K 38/12

(52) U.S. Cl. CPC C07D 233/88; A61K 31/4178; A61K 31/7048; A61K 38/12

(57) ABSTRACT

A new class of dimeric 2-aminoimidazole (2-AI) compounds that potentiate macrolide antibiotics against A. baumannii. A parent dimer lowers the MIC of clarithromycin (CLR) from 32 μg/mL to 1 μg/mL at a concentration of 7.5 μM (3.4 μg/mL), while a structure activity relationship (SAR) study on the dimeric 2-AI scaffold resulted in the identification of several compounds with increased activity. Substitution of fluorine on a central phenyl ring resulted in the most potent activity, with the lead compound, containing a fluorine ortho to each of the 2-AIs, that lowered the CLR MIC to 2 μg/mL against AB5075 at 1.5 μM (0.72 μg/mL), exceeding the activity of both the parent dimer and the lead aryl-2-AI. Furthermore, these dimeric 2-AI analogs exhibit favorably low mammalian cell toxicity.

### RELATED APPLICATIONS

This application claims priority under 35 U.S.C. § 119(e) to U.S. Provisional Patent Application No. 63/380,957, filed Oct. 26, 2022, which is incorporated herein by reference.

### GOVERNMENT SUPPORT

This invention was made with government support under Grant Nos. R01 AI136904 and R01 AI167284 awarded by the National Institutes of Health. The government has certain rights in the invention.

### BACKGROUND OF THE INVENTION

Antibiotic resistance remains one of the greatest threats to global human health. As of 2019, the Centers for Disease Control and Prevention (CDC) estimated that over 2 million people in the United States alone were infected with an antibiotic resistant bacterium each year, resulting in an approximately 35,000 deaths. The majority of those infections were caused by ESKAPE pathogens (Enterococcus faecium, Staphylococcus aureus, Klebsiella pneumoniae, Acinetobacter baumannii, Pseudomonas aeruginosa, and Enterobacter species), bacteria that are notorious for their ability to “escape” the action of commonly prescribed antibiotics. Of the ESKAPE pathogens, A. baumannii is a gram-negative bacteria that causes life-threatening infections and is now a leading cause of antibiotic-resistant infections worldwide. A. baumannii is especially problematic for patients in intensive care units and causes millions of infections per year across the globe, with mortality rates as high as 70%. Recent reports have indicated that A. baumannii is one of the most common coinfections with SARS-Cov-2, which has the potential to exacerbate the already considerable burden of A. baumannii infection. Increased use of antibiotics during the COVID-19 pandemic, particularly the macrolide azithromycin, will likely further contribute to the antibiotic resistance crisis.

The World Health Organization (WHO) has stated that the antibiotic resistant crisis is so serious that it threatens the advances of modern medicine, and that we are on the precipice of a post-antibiotic era, one in which common infections and/or minor injuries can routinely lead to increased patient mortality. Although concerted efforts to identify novel antibiotics remain crucial, progress has been slow, especially in the context of gram-negative bacteria, and resistance can develop rapidly. Therefore, a critical need exists for the exploration of alternative therapeutic approaches.

### SUMMARY

One alternative approach to combat antibiotic resistance is the development of antibiotic adjuvants. Adjuvant therapy consists of combinations of an antibiotic with a nonantibiotic compound that either inhibits resistant mechanisms directly or otherwise alters the physiology of an antibiotic-resistant cell to render it more susceptible to the antibiotic. Typically, macrolide antibiotics are prescribed to treat bacterial infections caused by gram-positive bacteria but have limited efficacy against gram-negative bacteria due to their inability to cross the outer membrane of the gram-negative cell. Despite this innate resistance, it has been shown that if the outer membrane has been compromised then macrolide antibiotics can become efficacious against gram-negative bacteria. For example, compounds that physically disrupt the outer membrane (OM), such as pentamidine and polymyxin derivatives, sensitize gram-negative bacteria to certain otherwise gram-positive-selective antibiotics.

Accordingly, this disclosure provides a compound of formula I:

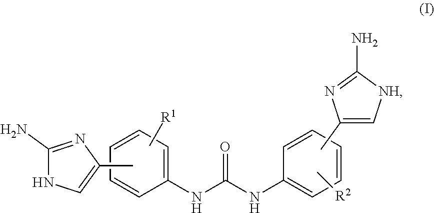

or a pharmaceutically acceptable salt thereof;
wherein,
   R¹ is halo, H, —(C₁-C₆)alkyl, or —O(C₁-C₆)alkyl; and
   R² is halo, H, —(C₁-C₆)alkyl, or —O(C₁-C₆)alkyl;
wherein at least one of R¹ or R² is not H, and each alkyl moiety is optionally substituted.

This disclosure also provides a combination comprising a compound described above and an antibiotic.

Additionally, this disclosure provides a method for potentiating an antibiotic against a bacterial infection, comprising administering an effective amount of the combination described above to a subject having a bacterial infection, wherein the compound potentiates the antibacterial activity of the antibiotic and thereby the combination treats the subject's bacterial infection.

The invention provides novel compounds of Formulas I-III, intermediates for the synthesis of compounds of Formulas I-III, as well as methods of preparing compounds of Formulas I-III. The invention also provides compounds of Formulas I-III that are useful as intermediates for the synthesis of other useful compounds. The invention provides for the use of compounds of Formulas I-III for the manufacture of medicaments useful for the treatment of bacterial infections in a mammal, such as a human.

The invention provides for the use of the compositions described herein for use in medical therapy. The medical therapy can be to treat an infection, for example, an infection caused by an ESKAPE pathogen. The invention also provides for the use of a composition as described herein for the manufacture of a medicament to treat an infection in a mammal, for example, A. baumannii in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Cytotoxicity data for compounds 1 and 2 against HepG2 cells.

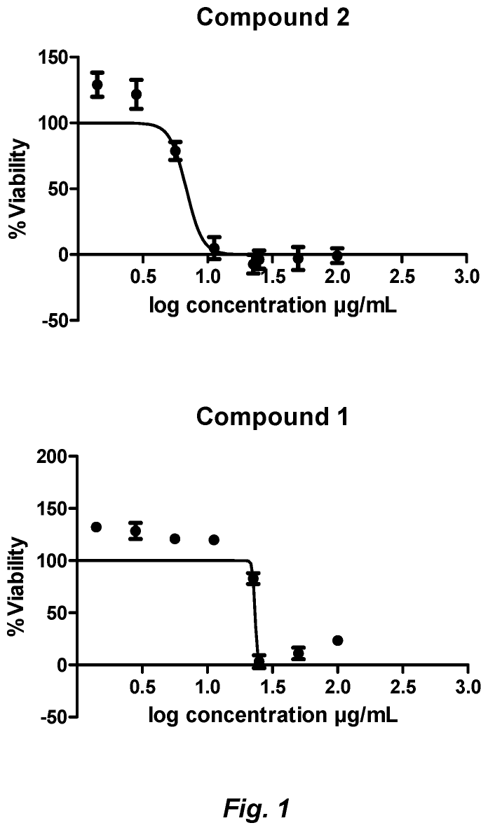

FIG. 2. Cytotoxicity data for compounds 9g, and 9k against HepG2 cells.

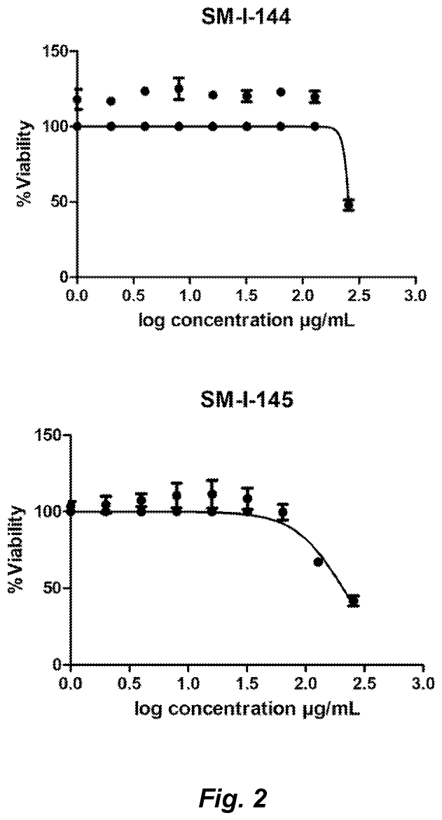

### DETAILED DESCRIPTION

According to the Centers for Disease Control and Prevention (CDC), hospital acquired infections have increased as much as 65% since 2019 due to the increased number of prolonged hospital visits. One of the main contributors to such infections is the gram-negative bacterium Acinetobacter baumannii. Our lab had reported several aryl 2-aminoimidazole (2-AI) analogs that potentiate macrolide antibiotics against A. baumannii. The lead compound decreased the clarithromycin (CLR) minimum inhibitory concentration (IC) against a highly virulent strain of A. baumannii, AB5075, by 128-fold, from 32 μg/mL to 0.25 μg/mL, at a concentration of 7.5 μM (3.0 μg/mL). Unfortunately, due to cytotoxicity concerns, these original compounds were not suitable for further development.

In this disclosure, we describe a new class of dimeric 2-aminoimidazole (2-AI) compounds that potentiate macrolide antibiotics against A. baumannii. The parent dimer lowers the MIC of CLR from 32 μg/mL to 1 μg/mL at a concentration of 7.5 μM (3.4 μg/mL), while a structure activity relationship (SAR) study on the dimeric 2-AI scaffold resulted in the identification of several compounds with increased activity. Substitution of fluorine on the central phenyl ring resulted in the most potent activity, with the lead compound, containing a fluorine ortho to each of the 2-AIs, that lowers the CLR MIC to 2 μg/mL against AB5075 at 1.5 μM (0.72 μg/mL), far exceeding the activity of both the parent dimer and the lead aryl-2-AI. Furthermore, these dimeric 2-AI analogs exhibit favorably low mammalian cell toxicity.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3- to 10-membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below. For example, a phenyl moiety or group may be substituted with one or more substituents RX where RX is at the ortho-, meta-, orpara-position, and X is an integer variable of 1 to 5.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring.

Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

The term “IC₅₀” is generally defined as the concentration required to inhibit a specific biological or biochemical function by half, or to kill 50% of the cells in a designated time period, typically 24 hours.

The compounds described herein, such as the compounds of formula I, potentiate the antibacterial activity of antibiotics such that the administering the combination of the compound and an antibiotic significantly enhances the treatment of a bacterial infection. As used herein, an “antibiotic” is a type of “biocide”. Common antibiotics include aminoglycosides, carbacephems (e.g., loracarbef), carbapenems, cephalosporins, glycopeptides (e.g., teicoplanin and vancomycin), macrolides, monobactams (e.g., aztreonam), penicillins, polypeptides (e.g., bacitracin, colistin, polymyxin B), quinolones, sulfonamides, tetracyclines, and the like. Antibiotics treat infections by either killing or preventing the growth of microorganisms. Many act to inhibit cell wall synthesis or other vital protein synthesis of the microorganisms. Several useful antibiotics, as well as methods of using them, that can be used in conjunction with the compounds described herein are described in US Publication Nos. 2022/0000118 (Melander) and 2023/0286922 (Melander), which are incorporated herein by reference.

### Embodiments of the Technology

1. A compound of formula I:

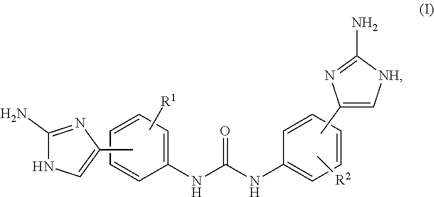

or a pharmaceutically acceptable salt thereof;
wherein,
   R¹ is halo, H, —(C₁-C₆)alkyl, or —O(C₁-C₆)alkyl; and
   R² is halo, H, —(C₁-C₆)alkyl, or —O(C₁-C₆)alkyl;
wherein at least one of R¹ or R² is not H, and each alkyl moiety is optionally substituted, for example, with one or more substituents as recited in the definition of ‘substituent’ herein.
   2. The compound of embodiment 1 wherein each 2-amino-1H-imidazolyl moiety is independently positioned meta or para relative to the urea moiety.
   3. The compound of embodiment 1 or 2 wherein one 2-amino-1H-imidazolyl moiety is positioned ortho or meta relative to R¹, and the second 2-amino-1H-imidazolyl moiety is positioned ortho or meta relative to R².
   4. The compound of any one of embodiments 1-3 wherein R¹ and R² are each independently F, Cl, H, CH₃, CF₃, or OCH₃.
   5. The compound of any one of embodiments 1-4 wherein R¹ and R² are both F.
   6. The compound of any one of embodiments 1-5 represented by formula (II):

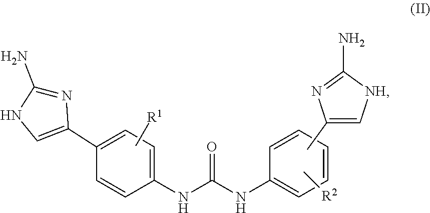

or a pharmaceutically acceptable salt thereof.
   7. The compound of any one of embodiments 1-5 represented by formula (III):

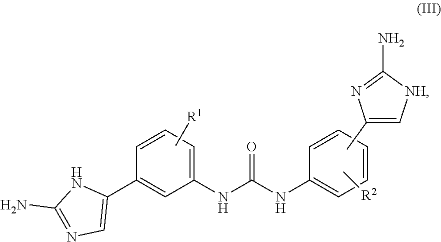

or a pharmaceutically acceptable salt thereof.
   8. The compound of any one of embodiments 1-7 wherein the compound is 9k, 9a, 9g, or a pharmaceutically acceptable salt thereof.
   9. The compound of any one of embodiments 1-7 wherein the compound is 9b, 9c, 9d, 9e, 9f, 9 h, 9i, 9j, 9l, 9m, or a pharmaceutically acceptable salt thereof.
   10. The compound of any one of embodiments 1-7 wherein the compound is 14a, 14b, 14c, or a pharmaceutically acceptable salt thereof.
   11. A combination comprising a compound according to any one of embodiments 1-10 and an antibiotic, and optionally a pharmaceutically acceptable carrier, diluent, or excipient.
   12. The combination of embodiment 11 wherein the compound is 1-(3-(2-amino-1H-imidazol-4-yl)-2-fluorophenyl)-3-(4-(2-amino-1H-imidazol-4-yl)-3-fluorophenyl)urea (9k).
   13. The combination of embodiment 11 or 12 wherein the antibiotic is a macrolide antibiotic.
   14. The combination of any one of embodiments 11-13 wherein the antibiotic is clarithromycin or colistin.
   15. A method for potentiating an antibiotic against a bacterial infection, comprising administering an effective amount of the combination according to any one of embodiments 11-14 to a subject having a bacterial infection, wherein the compound potentiates the antibacterial activity of the antibiotic and thereby the combination treats the subject's bacterial infection.
   16. The method of embodiment 15 wherein the compound is 1-(3-(2-amino-1H-imidazol-4-yl)-2-fluorophenyl)-3-(4-(2-amino-1H-imidazol-4-yl)-3-fluorophenyl)urea (9k).
   17. The method of embodiment 15 or 16 wherein the bacterial infection is caused by Enterococcus faecium, Staphylococcus aureus, Klebsiella pneumoniae, Acinetobacter baumannii, Pseudomonas aeruginosa, or an Enterobacter species.
   18. The method of any one of embodiments 15-17 wherein bacterial infection is a multi-drug resistant strain of Acinetobacter baumannii.
   19. The method of any one of embodiments 15-18 wherein the antibiotic is a Gram-negative antibiotic.
   20. The method of any one of embodiments 15-19 wherein the antibiotic is clarithromycin.

### Results and Discussion.

We previously investigated a series of aryl 2-aminoimidazole (2-AI) compounds (ACS Medicinal Chemistry Letters 2020 11 (9), 1723) for their ability to potentiate macrolide activity against multi-drug resistant (MDR) strains of A. baumannii and identified adjuvants 1 and 2 (Chart 1) as potential leads, with mechanistic studies indicating that activity was underpinned by altered lipooligosaccharide (LOS) biosynthesis and not direct disruption of the outer membrane. It was observed that at 7.5 mM (3.7 mg/mL), compound 1 reduced the clarithromycin minimum inhibitory concentration (MIC) 32-fold, from 32 mg/mL to 1.0 mg/mL against the highly virulent A. baumannii strain 5075 (AB5075), while at 5.0 mM, it reduced the clarithromycin MIC to 8.0 mg/mL. Compound 2, at 7.5 μM (3.0 μg/mL), reduced the clarithromycin MIC 128-fold to 0.25 μg/mL against AB5075 and at 5.0 mM, it reduced the clarithromycin MIC to 4.0 mg/mL.

Chart 1. Aryl-2AI and dimeric 2-AI compounds that potentiate clarithromycin in AB5075.

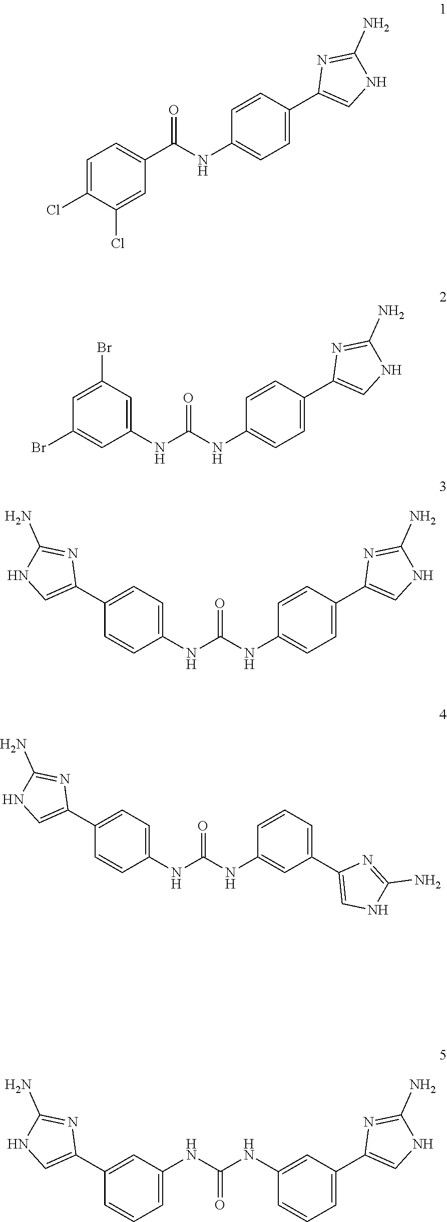 [SMILES: Nc1nc(-c2ccc(NC(=O)c3ccc(Cl)c(Cl)c3)cc2)c[nH]1 ; Nc1nc(-c2ccc(NC(=O)Nc3cc(Br)cc(Br)c3)cc2)c[nH]1 ; Nc1nc(-c2ccc(NC(=O)Nc3ccc(-c4c[nH]c(N)n4)cc3)cc2)c[nH]1 ; Nc1nc(-c2ccc(NC(=O)Nc3cccc(-c4c[nH]c(N)n4)c3)cc2)c[nH]1 ; Nc1nc(-c2cccc(NC(=O)Nc3cccc(-c4c[nH]c(N)n4)c3)c2)c[nH]1]

However, subsequent evaluation of 1 and 2 for mammalian cell toxicity using HepG2 cells returned IC₅₀ values of 16.9 mM and 47.6 μM, respectively. The therapeutic index (TI), in the case of conventional antibiotic development, is typically defined as defined as (mammalian cell IC₅₀)/(antibiotic MIC), with a TI of >50 desirable for further development. Because adjuvants are typically non-toxic, the aforementioned definition of TI is not applicable.

For adjuvant development, we define TI as (mammalian cell IC₅₀)/(adjuvant concentration that reduces the antibiotic MIC to breakpoint level). As macrolides are typically viewed as gram-positive selective antibiotics, there is no clinical breakpoint established for clarithromycin MICs against A. baumannii. For the few Gram-negative bacterial species where macrolide therapy is employed, breakpoint MICs are 1-8 mg/mL. In the absence of direct guidance, we have elected to use the clarithromycin breakpoint for Staphylococcus aureus as a guide. The Clinical Laboratory and Standards Institute (CLSI) currently defines the resistance breakpoint for clarithromycin in S. aureus as 2.0 mg/mL, thus compounds 1 and 2 returned TIs of 2.3-<3.4 and 6.3-<9.5 respectively. As these Tis are <<50, we precluded 1 and 2 for further development.

Based upon this low TI, we initiated a search for additional scaffolds that display high activity as adjuvants for macrolide potentiation, while showing significantly reduced cytotoxicity. Herein, we describe the identification and subsequent initial optimization of a series of dimeric 2-AI derivatives for their ability to potentiate macrolides, evaluation of leads for cytotoxicity, and mechanistic studies that establish activity is not dependent upon direct physical disruption of the outer membrane, unlike pentamidine and polymyxin derivatives.

We initiated this study by performing an in-depth screen of our internal library at 30 mM again using AB5075 as our representative bacterial strain and clarithromycin as our representative macrolide. Outside of the aryl-2-AIs that we had described previously, we identified dimeric compounds 3-5 as highly active clarithromycin adjuvants.

At 30 mM (13.4 mg/mL, Table 1) both the para-para linked dimer 3 and the para-meta linked dimer 4 suppressed the clarithromycin MIC to 0.125 mg/mL, a 256-fold reduction in comparison to clarithromycin alone. Compound 5, the meta-meta linked dimer, was the most active macrolide adjuvant we had discovered to date, returning an MIC of 0.03125 mg/mL (1024-reduction) at 30 mM. All three compounds were then resynthesized to confirm structure and samples with >95% purity (LCMS analysis) were retested for clarithromycin potentiation using AB5075. All three compounds showed identical activity to those from the library.

TABLE 1
Clarithromycin potentiation against AB5075 by compounds 1-5.

| | Compound | Concentration | Clarithromycin | |
|---|---|---|---|---|
| | MIC | Tested | MIC | Fold |
| Compound | (μM) | (μM) | (μg/mL) | Reduction |
| — | — | — | 32 | — |
| 1 | >200 | 30 | 0.5 | 64 |
| | | 7.5 | 1 | 32 |
| | | 5 | 8 | 4 |
| 2 | 50 | 15 | 0.125 | 256 |
| | | 7.5 | 0.25 | 128 |
| | | 5 | 4 | 8 |
| 3 | >200 | 30 | 0.125 | 256 |
| | | 20 | 0.5 | 64 |
| | | 10 | 4 | 8 |
| | | 7.5 | 8 | 4 |
| 4 | >200 μM | 30 | 0.125 | 256 |
| | (>447.1 | 20 | 1 | 32 |
| | μg/mL) | 10 | 16 | 2 |
| 5 | 200 | 30 | 0.03125 | 1024 |
| | (447.1 | 20 | 0.0625 | 512 |
| | μg/mL) | 10 | 0.5 | 64 |
| | | 7.5 | 1 | 32 |
| | | 5 | 4 | 8 |

Using newly synthesized material, we measured the inherent anti-bacterial activity of each dimer, with compounds 3 and 4 registering MICs against AB5075 of >200 mMv (>447.1 mg/mL), while compound 5's MIC was measured as 200 mMV (447.1 mg/mL), indicating that all three compounds were potentiating clarithromycin through a non-microbicidal mechanism. We also performed a dose-response study with each dimer (Table 1) and noted that compound 5 was the most potent, returning clarithromycin MICs of 1.0 and 4.0 mg/nmL at 7.5 μM (3.4 μg/mL) and 5.0 mM (2.2 μg/mL) respectively.

Encouraged by this initial activity, we evaluated the cytotoxicity of dimer 5 using an XTT assay with HepG2 cells to assess its potential as a lead. Compound 5 was found to be significantly less toxic than either 1 or 2, returning an IC₅₀ of 849.5 mM (380 μg/mL), thus returning a TI of 113-<170, indicating its potential to serve as a lead for further development.

With a lead scaffold in hand, we next sought to augment activity through analog synthesis. Previous studies on the aryl 2-AI scaffold (i.e., compounds 1/2) indicated that adjuvant activity could be optimized through strategic functional group placement (specifically fluorine) within the central aromatic ring and thus focused on modification of the corresponding aryl ring in the urea linked dimers. Based upon this plan, we developed dimers that were underpinned by dimerization of various 2-AI-derivatized anilines, with each 2-AI-derivatized aniline accessed through one of two synthetic routes depending on the commercial availability of the starting material (Scheme 1).

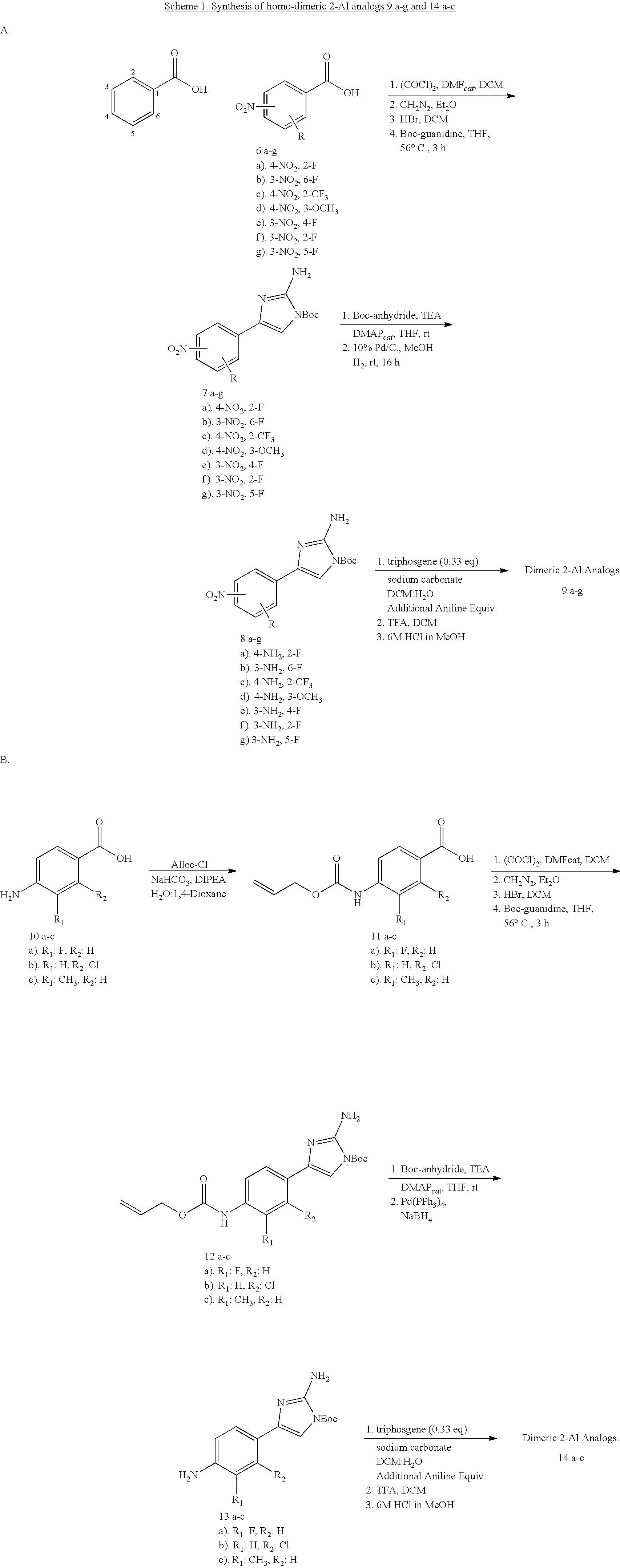

Synthetic route A began by conversion of commercially available 4- or 3-nitrobenzoic acid derivatives 6a-g to the corresponding acid chloride by treatment with oxalyl chloride, followed by subsequent reaction with diazomethane and quenching with hydrobromic acid to form the targeted a-bromo-ketones. Cyclization of each a-bromo-ketone with Boc-guanidine delivered 2-AIs 7a-g. Exhaustive boc-protection of the exocyclic amino group, followed by reduction of the nitro groups using 10% Pd/C and H₂ yielded anilines 8a-g for subsequent dimerization.

Route B began by following our previously reported procedure using commercially available 4-aminobenzoic acid derivatives 10a-c. Briefly, each amino group was protected using alloc chloride and the carboxylic acid was then transformed into the boc-protected 2-AIs 12a-c using the identical four-step approach as above. Protection of the exocyclic amino group of the 2-AI with Boc anhydride, followed by removal of the alloc protecting group using palladium tetrakis(triphenylphosphine) and sodium borohydride, yielded aniline derivatives 13a-c.

For simplicity, our first library focused on evaluation of the corresponding homo-dimers, thus each 2-AI-aniline was subjected to dimerization using triphosgene, which followed by boc-deprotection with trifluoroacetic acid and counter salt exchange delivered urea linked 2-AI dimers 9a-g and 14a-c (Chart 2).

Chart 2. Homo-dimeric 2-AI analogs synthesized.

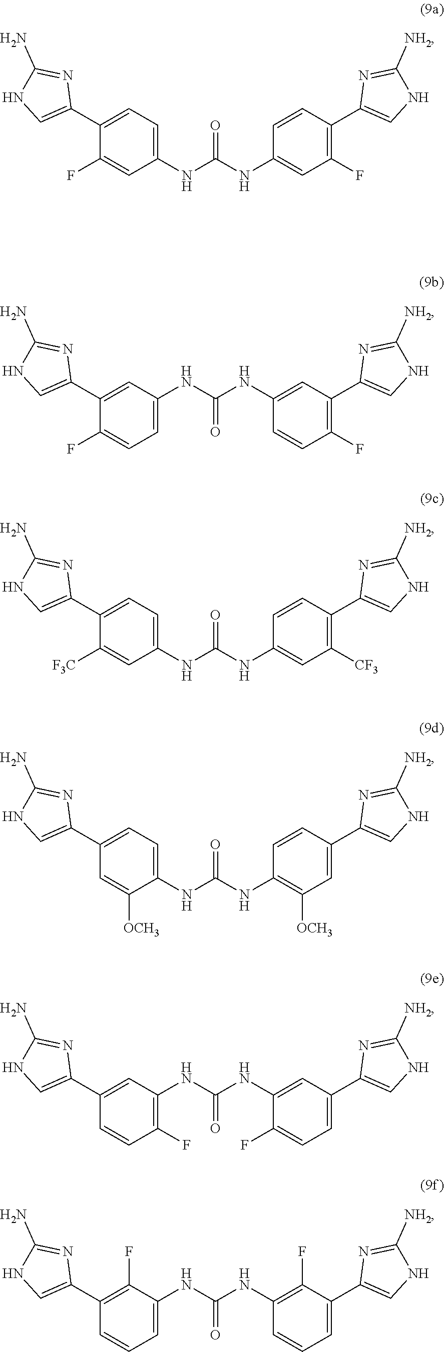 [SMILES: Nc1nc(-c2ccc(NC(=O)Nc3ccc(-c4c[nH]c(N)n4)c(F)c3)cc2F)c[nH]1 ; Nc1nc(-c2cc(NC(=O)Nc3ccc(F)c(-c4c[nH]c(N)n4)c3)ccc2F)c[nH]1 ; Nc1nc(-c2ccc(NC(=O)Nc3ccc(-c4c[nH]c(N)n4)c(C(F)(F)F)c3)cc2C(F)(F)F)c[nH]1 ; COc1cc(-c2c[nH]c(N)n2)ccc1NC(=O)Nc1ccc(-c2c[nH]c(N)n2)cc1OC ; Nc1nc(-c2ccc(F)c(NC(=O)Nc3cc(-c4c[nH]c(N)n4)ccc3F)c2)c[nH]1 ; Nc1nc(-c2cccc(NC(=O)Nc3cccc(-c4c[nH]c(N)n4)c3F)c2F)c[nH]1]

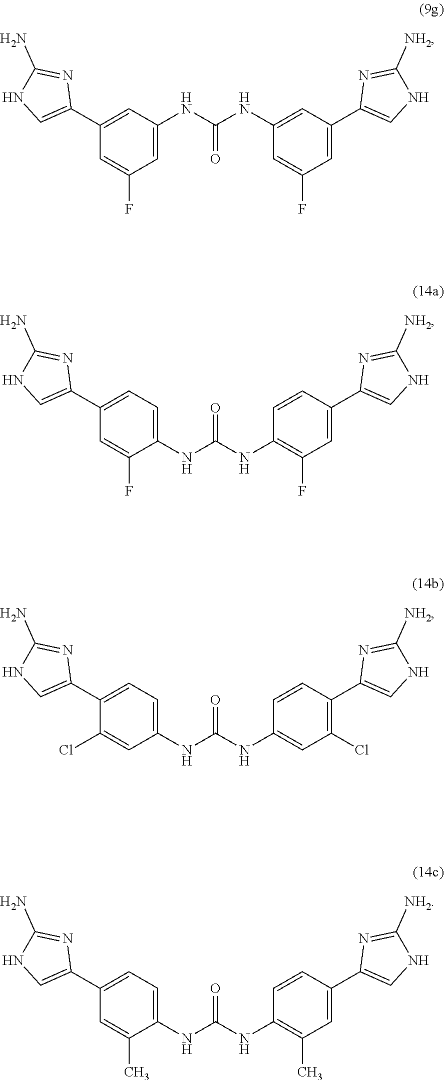 [SMILES: Nc1nc(-c2cc(F)cc(NC(=O)Nc3cc(F)cc(-c4c[nH]c(N)n4)c3)c2)c[nH]1 ; Nc1nc(-c2ccc(NC(=O)Nc3ccc(-c4c[nH]c(N)n4)cc3F)c(F)c2)c[nH]1 ; Nc1nc(-c2ccc(NC(=O)Nc3ccc(-c4c[nH]c(N)n4)c(Cl)c3)cc2Cl)c[nH]1 ; Cc1cc(-c2c[nH]c(N)n2)ccc1NC(=O)Nc1ccc(-c2c[nH]c(N)n2)cc1C]

Dimers were first analyzed for standalone toxicity against AB5075. Each dimeric 2-AI derivative was essentially non-toxic, returning MICs of ≥200 μM (Tables 2 and 3). The MIC of clarithromycin was then determined against AB5075 in both the absence and presence of each compound at an initial concentration of 30 μM (Tables 2 and 3).

To begin the SAR study, symmetrical dimers 9a and 9b with fluorine substitution at the 2,2′ or 6,6′ positions on the phenyl core were synthesized first (numbering scheme as outlined below).

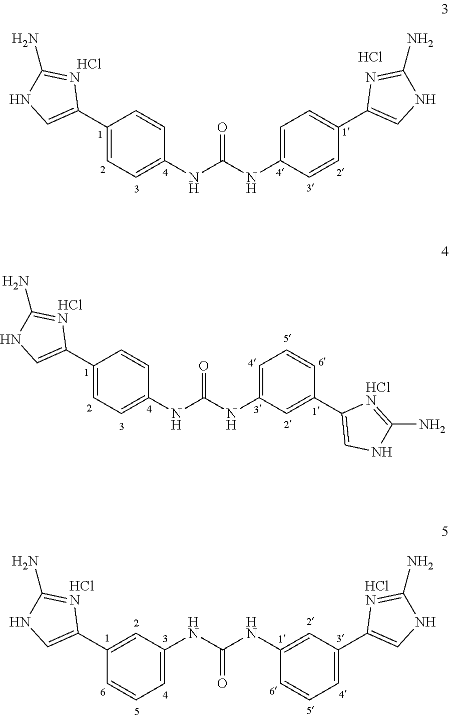

Both 9a and 9b displayed CLR potentiation activity against AB5075 at 30 μM that was comparable to the lead underivatized dimer 5 (Table 2). The para-para fluorinated dimer 9a was more potent than the meta-meta fluorinated dimer 9b, lowering the CLR MIC by 512-fold at 30 μM, compared to 256-fold, and so we elected to first evaluate additional para-para-analogs. We found that incorporation of substituents larger than a fluoro-substituent, including chloro- (14b), methyl- (14c), trifluoromethyl- (9c), or methoxy- (9d) at either the 2- or 3-positions of the phenyl rings abolished all activity, suggesting that effects are the likely cause for loss of activity, since neither electron donating nor electron withdrawing groups (outside of fluorine) displayed significant activity. Moving the fluorines from the 2, 2′ to the 3,3′ position (14a) also led to a decrease in activity, returning a clarithromycin MIC of 4.0 mg/mL at 30 mM.

TABLE 2
CLR potentiation by homo-dimers 9a-g and 14a-c against AB5075.

| | Compound | Concentration | CLR | Fold |
|---|---|---|---|---|
| Adjuvant | MIC (μM) | Tested (μM) | MIC (μg/mL) | Reduction |
| — | — | — | 32 | — |
| 9a | >200 | 30 | 0.03125 | 1024 |
| 9b | >200 | 30 | 0.125 | 256 |
| 9c | >200 | 30 | 2 | 16 |
| 9d | 200 | 30 | 8 | 4 |
| 9e | >200 | 30 | 32 | 1 |
| 9f | >200 | 30 | 0.25 | 128 |
| 9g | >200 | 30 | ≤0.0625 | ≥512 |
| 14a | >200 | 30 | 4 | 8 |
| 14b | >200 | 30 | 32 | 1 |
| 14c | >200 | 30 | 32 | 1 |

Because fluorination appeared to be a promising substitution choice for adjuvant activity, the next set of analogs synthesized were the meta-meta dimers where we systematically incorporated fluorine to access all possible fluorinated homodimer (9e-g, Chart 2). Compound 9g (5.5′-difluoro) displayed greater activity at 30 μM than dimers that contained fluorines at either the 2,2′- (9f),4,6′-(9e) or 6,4′-positions (9b) of the ring and displayed comparable activity to the parent meta-meta-dimer 5 at 30 μM, reducing CLR's MIC more than 512-fold (Table 1 and 2).

The last set of analogs we evaluated was a series of unsymmetrical 2-AI dimers. The design of the unsymmetrical dimers 9h-m (Chart 3) was based on the activity seen with the homodimers where strategic fluorine incorporation enhanced activity. With the exception of compound 9i, all heterodimers suppressed the clarithromycin MIC below 2.0 mg/mL, with compounds 9j-9m all returning clarithromycin MICs ≤0.0625 mg/mL (Table 3).

Chart 3. Hetero-dimeric 2-AI analogs synthesized.

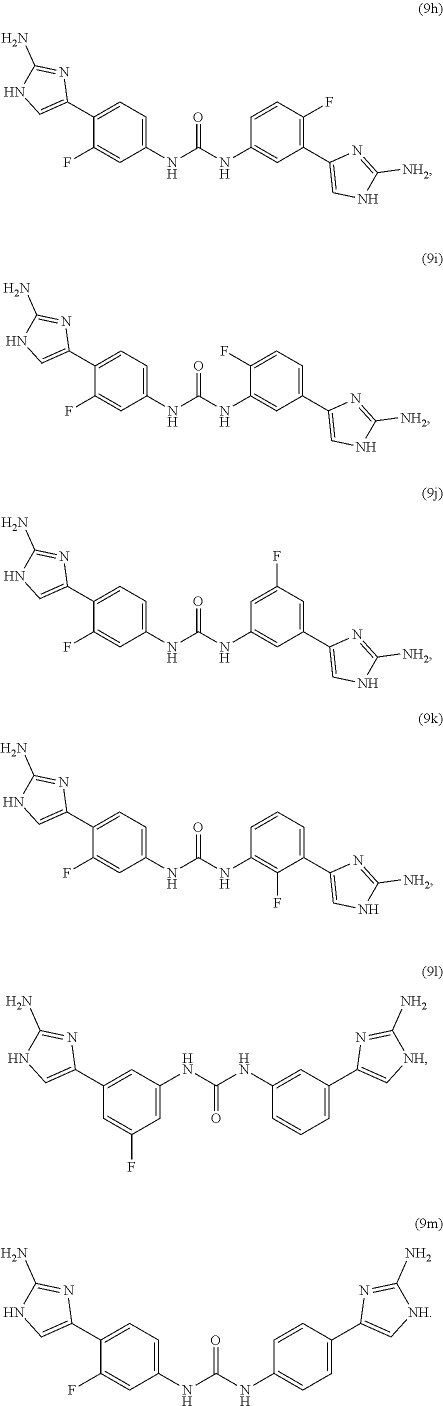 [SMILES: Nc1nc(-c2ccc(NC(=O)Nc3ccc(F)c(-c4c[nH]c(N)n4)c3)cc2F)c[nH]1 ; Nc1nc(-c2ccc(F)c(NC(=O)Nc3ccc(-c4c[nH]c(N)n4)c(F)c3)c2)c[nH]1 ; Nc1nc(-c2cc(F)cc(NC(=O)Nc3ccc(-c4c[nH]c(N)n4)c(F)c3)c2)c[nH]1 ; Nc1nc(-c2ccc(NC(=O)Nc3cccc(-c4c[nH]c(N)n4)c3F)cc2F)c[nH]1 ; Nc1nc(-c2cccc(NC(=O)Nc3cc(F)cc(-c4c[nH]c(N)n4)c3)c2)c[nH]1 ; Nc1nc(-c2ccc(NC(=O)Nc3ccc(-c4c[nH]c(N)n4)c(F)c3)cc2)c[nH]1]

TABLE 3
CLR potentiation by hetero-dimers 9h-m against AB5075.

| | Compound | Concentration | CLR | Fold |
|---|---|---|---|---|
| Adjuvant | MIC (μM) | Tested (μM) | MIC (μg/mL) | Reduction |
| — | — | — | 32 | — |
| 9h | >200 | 30 | 0.5 | 64 |
| 9i | >200 | 30 | 8 | 4 |
| 9j | >200 | 30 | 0.0625 | 512 |
| 9k | >200 | 30 | ≤0.0625 | ≥512 |
| 9l | >200 | 30 | 0.0625 | 512 |
| 9m | >200 | 30 | 0.0625 | 512 |

After this initial survey of activity at a fixed concentration (30 M), a dose response study was conducted with the nine most active compounds (9a-b, 9f-h, 9j-m,Table 4), where we sought to define the lowest concentration at which the adjuvant suppressed the clarithromycin MIC to 2.0 mg/mL. Eight of the nine compounds tested returned clarithromycin MICs of ≤2.0 mg/mL at 7.5 μM, with 9h delivering a clarithromycin MIC of 4.0 mg/mL at 20 mM. Compounds 9f, 9l, and 9m achieved the 2.0 mg/mL metric at 7.5 mM, while compounds 9a, 9b, and 9j did so at 5 mM. The two most active compounds, 9g and 9k, where the most potent dimers in the series, active at 2.5 mM and 1.5 mM (0.72 μg/mL) respectively. To our knowledge, 9g and 9k are the most potent A. baumannii macrolide adjuvants disclosed to date.

TABLE 4
Dose response for most active dimers against AB5075.

| | Concentration | CLR | Fold |
|---|---|---|---|
| Adjuvant | Tested (μM) | MIC (μg/mL) | Reduction |
| CLR | — | 32 | — |
| 9a | 20 | 0.0625 | 512 |
| | 10 | 0.125 | 256 |
| | 7.5 | 0.5 | 64 |
| | 5 | 2 | 16 |
| 9b | 20 | 0.25 | 128 |
| | 10 | 0.5 | 64 |
| | 7.5 | 1 | 32 |
| | 5 | 2 | 16 |
| 9f | 20 | 0.5 | 64 |
| | 10 | 1 | 32 |
| | 7.5 | 2 | 16 |
| 9g | 20 | 0.0625 | 512 |
| | 10 | 0.125 | 256 |
| | 7.5 | 0.25 | 128 |
| | 5 | 0.5 | 64 |
| | 2.5 | 1 | 32 |
| 9h | 20 | 4 | 8 |
| 9j | 20 | 0.125 | 256 |
| | 10 | 0.125 | 256 |
| | 7.5 | 0.25 | 128 |
| | 5 | 1 | 32 |
| 9k | 20 | ≤0.0625 | ≥512 |
| | 10 | ≤0.0625 | ≥512 |
| | 7.5 | ≤0.0625 | ≥512 |
| | 5 | 0.0625 | 512 |
| | 2.5 | 0.125 | 256 |
| | 1.5 | 2 | 16 |
| 9l | 20 | 0.0625 | 512 |
| | 10 | 0.5 | 64 |
| | 7.5 | 1 | 32 |
| 9m | 20 | 0.5 | 64 |
| | 10 | 1 | 32 |
| | 7.5 | 2 | 16 |

Upon completion of the dose response, the most active dimers from each class, 9a, 9g, and 9k, were then tested against a panel of A. baumannii isolates that encompass all major and most minor clinically relevant clades (Table 5). The standalone clarithromycin IVIC for each of the strains was measured first, with certain strains returning a clarithromycin MIC of 64 mg/mL, other strains registering a clarithromycin MIC of 16 mg/mL, and the rest displaying a clarithromycin MIC of 32 mg/mL. All three compounds suppressed macrolide resistance in 25 AB strains at 10 μM (4.8 μg/mL) or less, lowering CLR MIC values by at least four-fold. Compounds 9a and 9g alone had no toxicity toward any of the panel of isolates, however compound 9k displayed some toxicity toward several of the AB isolates with MICs of 100, 50 and 25 μM, respectively.

TABLE 5
Compounds in combination with colistin against AB 5075.

| | Compound MIC | Concentration | Colistin |
|---|---|---|---|
| Adjuvant | Alone in AB 5075 (μM) | Tested (μM) | MIC (μg/mL) |
| Colistin | — | — | 1 |
| 3 | >200 | 30 | 2 |
| 4 | >200 | 30 | 0.25 |
| 5 | 200 | 30 | 2 |
| 9k | >200 | 30 | 2 |

We then examined the activity of compounds 9a, 9g, and 9k, as well as the three original dimers 3, 4 and 5, in combination with two additional macrolides: azithromycin (AZM) and erythromycin (ERM) as well as the rifamycin rifampin (RIF) against AB5075. At ≤30% their MIC, all six dimers displayed significant activity with all three additional antibiotics. Compounds 9a, 9g and 9k displayed greater potentiation activity at lower concentrations than their corresponding parent dimer, decreasing both the AZM and ERY MICs by at least 16-fold and the RIF MIC by 32-fold at 10 μM. Additionally, compound 9k remained the most active adjuvant in combination with these other antibiotics, decreasing the AZM and ERY MICs by at least 64-fold at a concentration of 7.5 μM (3.6 μg/mL), and the RIF MIC by 32-fold at a concentration of 5 μM (2.4 μg/mL).

Our previously macrolide adjuvants based on the 2-AI-aryl framework (i.e., compounds 1 and 2) were found to antagonize the activity of colistin against AB 5075, causing an increase of its MIC from 1 to 4 μg/mL at 30 μM (ACS Infect. Dis. 2019, 5 (7), 1223). Colistin's mechanism is dependent on binding to the lipooligosaccharide (LOS) portion A. baumannii 's outer membrane (A. baumannii does not contain O-antigen in its lipopolysaccharide (LPS) and this molecule is thus referred to as LOS), and certain strains of A. baumannii (including AB5075) are able to become LOS deficient to achieve colistin resistance. However, dropping LOS comes with fitness costs such as reduced growth rates, lower virulence, and increased sensitivity towards certain antibiotics.

Based upon the observed colistin antagonism, it was posited that these aryl 2-AI adjuvants may be potentiating CLR and other macrolides by inhibiting LOS assembly or production. To test whether these dimeric 2-AI compounds may have a similar affect as the aryl 2-AI's, we measured the MIC of colistin in the absence or presence of four 2-AI dimers at 30 mM: the original three parent dimers 3-5 and the most active dimer 9K. combination with colistin. (Table 5). Colstin alone registered an MIC of 1.0 mg/mL, and compounds 3, 4, and 9k all caused the colistin MIC to shift to 2.0 mg/mL. Interestingly, compound 4 synergized with colistin and repressed the MIC to 0.25 mg/mL.

To probe the spectrum of activity of these compounds outside A. baumannii we tested compound 9k in combination with CLR among three other gram-negative bacterial species, K. pneumoniae (strain KP 2146), P. aeruginosa (PAO1), and Escherichia coli (EC 25. Compound 9k displayed no standalone toxicity toward both KP2146 and PAO1 (MICs >200 μM), however it did exhibit modest toxicity toward EC25922 (MIC 12.5 μM). Compound 9k potentiated CLR in all three gram-negative species at least 256-fold (Table 6). We also examined the activity of 9a, 9g, and 9k in combination with RIF against K. pneumoniae 2146. Again, we saw greater potentiation activity at lower concentrations with these compounds than with the parent dimers. However, there was significantly more activity seen overall against A. baumannnii than with KP 2146.

TABLE 6
Compound 9k alone and in combination with
CLR against other Gram-negative species.

| | CLR | Compound | Concentration | CLR |
|---|---|---|---|---|
| Gram (—) | MIC | MIC | Tested | MIC |
| Strain | (μg/mL) | (μM) | (μM) | (μg/mL) + 9k |
| KP 2146 | 512 | >200 | 30 | 1 |
| | | | 10 | 2 |
| | | | 7.5 | 16 |
| PAO1 | 256 | >200 | 30 | 0.5 |
| | | | 20 | 4/8 |
| EC 25922 | 32 | 12.5 | 3.75 | 0.125 |
| | | | 3.0 | 4 |
| | | | 2.5 | 32 |

We next probed whether the CLR adjuvant activity of the lead dimers was dependent upon outer membrane disruption. First, we examined the impact that addition of MgCl₂ had on activity. Divalent cations, such as Mg²⁺, are known to stabilize the outer membrane of gram-negative bacteria and the activity of membrane disruptors are known to be muted/abrogated by the addition of such cations. When the media was supplemented with MgCl₂ (20 mM final concentration), activity was essentially abolished. As suppression of activity could conceivably originate from either blocking the compound's mechanism of action or simply making the membrane more impermeable that leads to the inability of the compound to penetrate the outer membrane, we next studied the impact that the addition of exogenous LOS had upon activity. If these compounds are indeed acting as general membrane perturbant to achieve CLR potentiation, the addition of LOS should compete for compound binding and lead to abrogation of activity. LOS was isolated from AB5075 and when added to the media at either 0.2 mg/mL or 0.5 mg/mL, no loss of adjuvant activity was noted for 9k or 9a, indicating that LOS binding is not involved in the mechanism by which these dimers sensitize A. baumannii to CLR.

We then quantified how compound 9k impacted membrane integrity using the BacLight assay. Compound 9k had essentially no impact on the membrane in comparison to untreated control and produced a −1.2±2.2% change in membrane permeability. Taken together, the mechanism by which these dimers achieve CLR potentiation is not dependent upon targeting and disrupting the bacterium's outer membrane.

Finally, to establish the TI of these dimers, we interrogated our set of analogs for host cell toxicity using embryonic HepG2 cells. Focusing on the most active fluorinated dimers and comparing them to the lead aryl-2AI analogs, half-maximal inhibition activity (IC₅₀) values were calculated. As delineated above, compounds 1 and 2 displayed moderate to high toxicity, exhibiting IC₅₀ values of 23.2±0.4 μg/mL (47.6 μM) and 6.8±0.8 μg/mL (16.9 μM) respectively. Lead compounds 9g (SM-I-144) and 9k (SM-I-145) however, exhibit much more favorable cytotoxicity against HepG2 cells, returning IC₅₀ values >256 μg/mL (>530 μM) and 208.1±15.6 μg/mL (430.6 μM), and TI's of >212 and 287 respectively.

Additionally, dimer compounds 5, 9a, and 9k displayed favorable metabolic and plasma stability maintaining a steady concentration over 60 minutes for microsomal stability and a steady concentration over 4 hours for plasma stability. Furthermore, the hemolysis assay showed little to no lytic effect when increasing concentrations of compound 9k from 25 to 400 M, and compound 9k at 10 μM in combination with 0.0625 μg/mL clarithromycin are dosed with red blood cells when compared to the 1% Triton X sample as the 100% lysis control.

In conclusion, there is no question that MDR A. baumannii infections cause a serious burden on the health care system and novel approaches to eradicate such infections are greatly needed. Here we have identified and synthesized a series of novel 2-AI dimeric analogs for the potentiation of clarithromycin against a highly virulent strain of A. baumannii, 5075, as well as 25 additional AB isolates. The SAR study revealed the dimeric 2-AI compound 9k to be the lead adjuvant, which enhanced clarithromycin activity by 16-fold at a concentration as low at 1.5 μM. Compound 9k also sensitized AB5075 to several additional antibiotics including, azithromycin, erythromycin, and rifampin but did not potentiate the activity of the membrane-active antibiotic colistin. Because all the antimicrobials that were potentiated by 9k must penetrate through the outer membrane of the gram-negative cell to be effective, our dimer compounds appear to affect membrane integrity to enhance macrolide activity in A. baumannii.

Several studies have been conducted to show that they are not directly binding to the LPS itself, however further mechanistic studies are currently ongoing to address whether they are affecting assembly or biosynthesis of LPS. Furthermore, 2-AI dimer 9k potentiated CLR against several other gram-negative pathogens, further highlighting its potential as an adjuvant. Additionally, these dimeric 2-AI analogs exhibited lower mammalian toxicity obtaining IC₅₀ values >200 μg/mL in HepG2 cells, as well as favorable metabolic and plasma stability with no lytic affect.

### General Synthetic Methods.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

Generally, the reaction conditions such as temperature, reaction time, solvents, work-up procedures, and the like, will be those common in the art for the particular reaction to be performed. The cited reference material, together with material cited therein, contains detailed descriptions of such conditions. Typically, the temperatures will be −100° C. to 200° C., solvents will be aprotic or protic depending on the conditions required, and reaction times will be 1 minute to 10 days. Work-up typically consists of quenching any unreacted reagents followed by partition between a water/organic layer system (extraction) and separation of the layer containing the product.

Oxidation and reduction reactions are typically carried out at temperatures near room temperature (about 20° C.), although for metal hydride reductions frequently the temperature is reduced to 0° C. to −100° C. Heating can also be used when appropriate. Solvents are typically aprotic for reductions and may be either protic or aprotic for oxidations. Reaction times are adjusted to achieve desired conversions.

Condensation reactions are typically carried out at temperatures near room temperature, although for non-equilibrating, kinetically controlled condensations reduced temperatures (0° C. to −100° C.) are also common. Solvents can be either protic (common in equilibrating reactions) or aprotic (common in kinetically controlled reactions). Standard synthetic techniques such as azeotropic removal of reaction by-products and use of anhydrous reaction conditions (e.g. inert gas environments) are common in the art and will be applied when applicable.

Protecting Groups. The term “protecting group” refers to any group which, when bound to a hydroxy or other heteroatom prevents undesired reactions from occurring at this group and which can be removed by conventional chemical or enzymatic steps to reestablish the hydroxyl group. The particular removable protecting group employed is not always critical and preferred removable hydroxyl blocking groups include conventional substituents such as, for example, allyl, benzyl, acetyl, chloroacetyl, thiobenzyl, benzylidene, phenacyl, methyl methoxy, silyl ethers (e.g., trimethylsilyl (TMS), t-butyl-diphenylsilyl (TBDPS), or t-butyldimethylsilyl (TBS)) and any other group that can be introduced chemically onto a hydroxyl functionality and later selectively removed either by chemical or enzymatic methods in mild conditions compatible with the nature of the product.

Suitable hydroxyl protecting groups are known to those skilled in the art and disclosed in more detail in T. W. Greene, Protecting Groups In Organic Synthesis; Wiley: New York, 1981 (“Greene”) and the references cited therein, and Kocienski, Philip J.; Protecting Groups (Georg Thieme Verlag Stuttgart, New York, 1994), both of which are incorporated herein by reference.

Protecting groups are available, commonly known and used, and are optionally used to prevent side reactions with the protected group during synthetic procedures, i.e. routes or methods to prepare the compounds by the methods of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis.

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or iniection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compounds described herein can be effective as an adjuvant for an antibiotic and have higher potency and/or reduced toxicity compared to only the antibiotic.

The invention provides therapeutic methods of treating a bacterial infection in a mammal, which involve administering to a mammal having cancer an effective amount of a compound, combination, or composition described herein. A mammal includes a primate, human, rodent, canine, feline, bovine, ovine, equine, swine, caprine, bovine and the like.

The ability of a compound of the invention to treat an infection may be determined by using assays well known to the art. For example, the design of treatment protocols, toxicity evaluation, data analysis, and quantification of cell kill.

The following Example is intended to illustrate the above invention and should not be construed as to narrow its scope. One skilled in the art will readily recognize that the Example suggest many other ways in which the invention could be practiced. It should be understood that numerous variations and modifications may be made while remaining within the scope of the invention.

### EXAMPLE

### Example 1. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| | mg/tablet |
|---|---|
| (i) Tablet 1 | |
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | |
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| | mg/mL |
|---|---|
| (iv) Injection 1 (1 mg/mL) | |
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/mL) | |
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| | wt. % |
|---|---|
| (vii) Topical Gel 1 | |
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |
| (viii) Topical Gel 2 | |
| ‘Compound X’ | 5% |
| Methylcellulose | 2% |
| Methyl paraben | 0.2% |
| Propyl paraben | 0.02% |
| Purified water | q.s. to 100 g |
| (ix) Topical Ointment | |
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |
| (x) Topical Cream 1 | |
| ‘Compound X’ | 5% |
| White bees wax | 10% |
| Liquid paraffin | 30% |
| Benzyl alcohol | 5% |
| Purified water | q.s. to 100 g |
| (xi) Topical Cream 2 | |
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. The formulations can further include an antibiotic in the same amount as Compound X, or 5% to 95% of the amount of Compound X, or 2-5 times the amount of Compound X. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A compound of formula I: 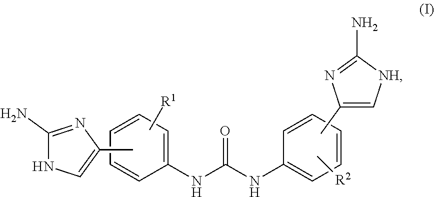
or a pharmaceutically acceptable salt thereof,
wherein,
R¹ is halo, H, —(C₁-C₆)alkyl, or —O(C₁-C₆)alkyl; and
R² is halo, H, —(C₁-C₆)alkyl, or —O(C₁-C₆)alkyl;
wherein at least one of R¹ or R² is not H, and each alkyl moiety is optionally substituted.

2. The compound of claim 1 wherein each 2-amino-1H-imidazolyl moiety is independently positioned meta or para relative to the urea moiety.

3. The compound of claim 1 wherein one 2-amino-1H-imidazolyl moiety is positioned ortho or meta relative to R¹, and the second 2-amino-1H-imidazolyl moiety is positioned ortho or meta relative to R².

4. The compound of claim 1 wherein R¹ and R² are each independently F, Cl, H, CH₃, CF₃, or OCH₃.

5. The compound of claim 1 wherein R¹ and R² are both F.

6. The compound of claim 1 represented by formula (II): 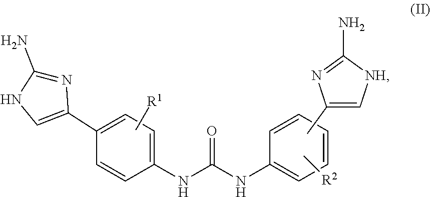
or a pharmaceutically acceptable salt thereof.

7. The compound of claim 1 represented by formula (III): 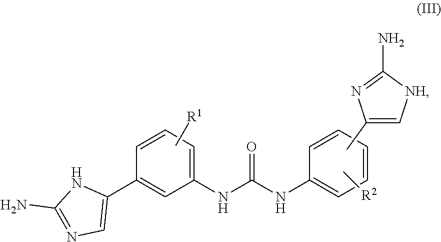
or a pharmaceutically acceptable salt thereof.

8. The compound of claim 1 wherein the compound is: 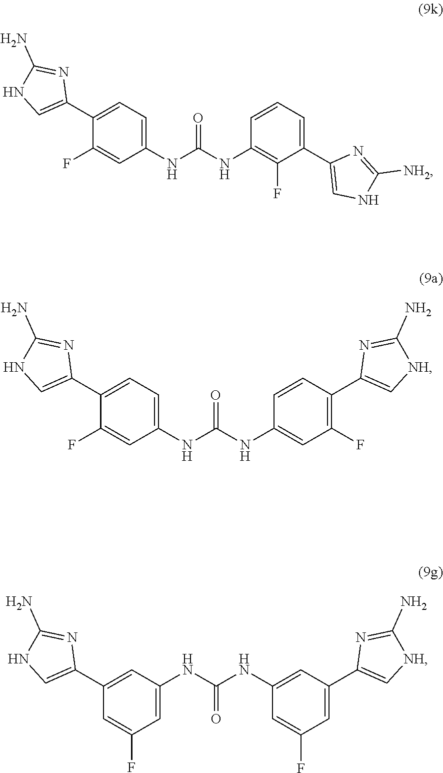 [SMILES: Nc1nc(-c2ccc(NC(=O)Nc3cccc(-c4c[nH]c(N)n4)c3F)cc2F)c[nH]1 ; Nc1nc(-c2ccc(NC(=O)Nc3ccc(-c4c[nH]c(N)n4)c(F)c3)cc2F)c[nH]1 ; Nc1nc(-c2cc(F)cc(NC(=O)Nc3cc(F)cc(-c4c[nH]c(N)n4)c3)c2)c[nH]1]
or a pharmaceutically acceptable salt thereof.

9. The compound of claim 1 wherein the compound is: 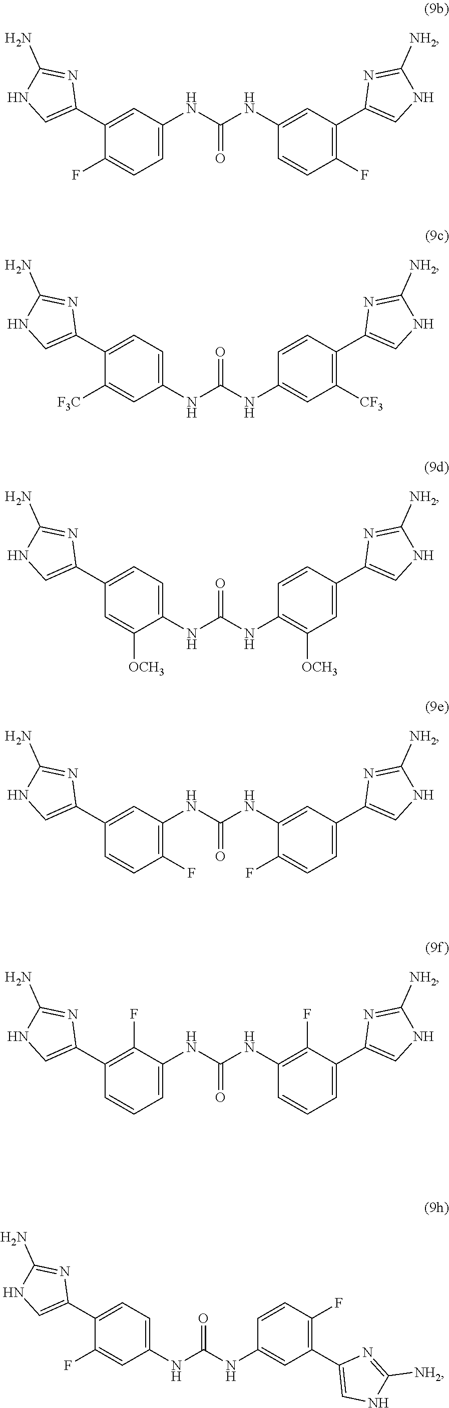 [SMILES: Nc1nc(-c2cc(NC(=O)Nc3ccc(F)c(-c4c[nH]c(N)n4)c3)ccc2F)c[nH]1 ; Nc1nc(-c2ccc(NC(=O)Nc3ccc(-c4c[nH]c(N)n4)c(C(F)(F)F)c3)cc2C(F)(F)F)c[nH]1 ; COc1cc(-c2c[nH]c(N)n2)ccc1NC(=O)Nc1ccc(-c2c[nH]c(N)n2)cc1OC ; Nc1nc(-c2ccc(F)c(NC(=O)Nc3cc(-c4c[nH]c(N)n4)ccc3F)c2)c[nH]1 ; Nc1nc(-c2cccc(NC(=O)Nc3cccc(-c4c[nH]c(N)n4)c3F)c2F)c[nH]1 ; Nc1nc(-c2ccc(NC(=O)Nc3ccc(F)c(-c4c[nH]c(N)n4)c3)cc2F)c[nH]1] 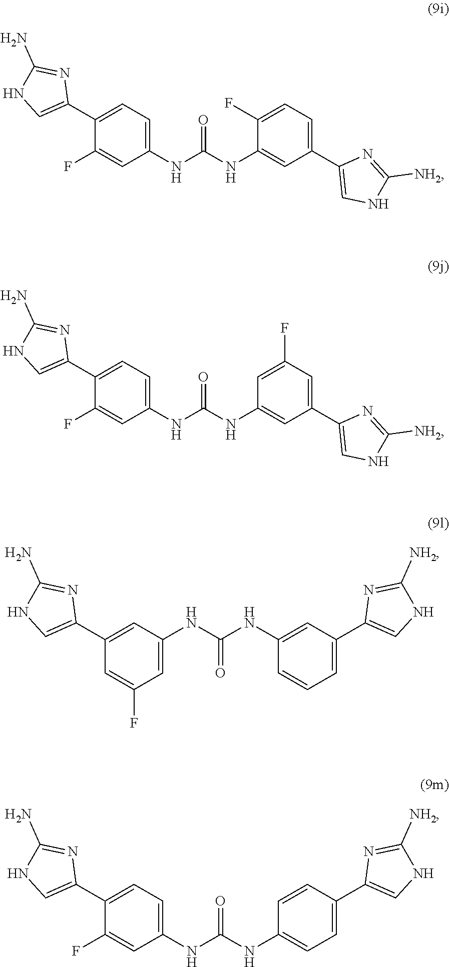 [SMILES: Nc1nc(-c2ccc(F)c(NC(=O)Nc3ccc(-c4c[nH]c(N)n4)c(F)c3)c2)c[nH]1 ; Nc1nc(-c2cc(F)cc(NC(=O)Nc3ccc(-c4c[nH]c(N)n4)c(F)c3)c2)c[nH]1 ; Nc1nc(-c2cccc(NC(=O)Nc3cc(F)cc(-c4c[nH]c(N)n4)c3)c2)c[nH]1 ; Nc1nc(-c2ccc(NC(=O)Nc3ccc(-c4c[nH]c(N)n4)c(F)c3)cc2)c[nH]1]
or a pharmaceutically acceptable salt thereof.

10. The compound of claim 1 wherein the compound is: 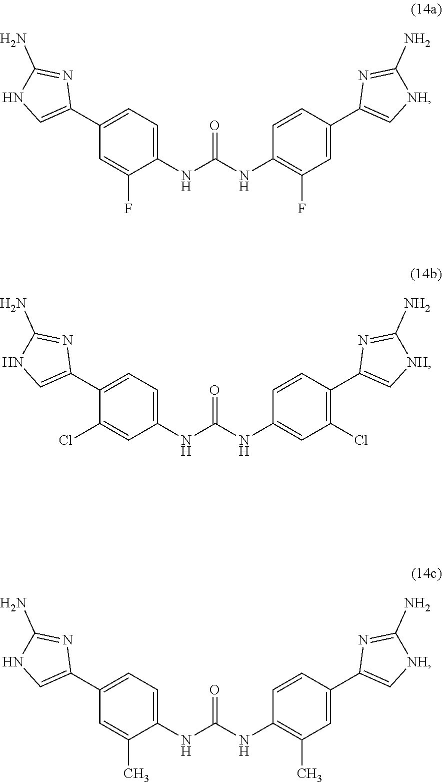 [SMILES: Nc1nc(-c2ccc(NC(=O)Nc3ccc(-c4c[nH]c(N)n4)cc3F)c(F)c2)c[nH]1 ; Nc1nc(-c2ccc(NC(=O)Nc3ccc(-c4c[nH]c(N)n4)c(Cl)c3)cc2Cl)c[nH]1 ; Cc1cc(-c2c[nH]c(N)n2)ccc1NC(=O)Nc1ccc(-c2c[nH]c(N)n2)cc1C]
or a pharmaceutically acceptable salt thereof.

11. A composition comprising a compound according to claim 1 and an antibiotic.

12. The composition of claim 11 wherein the compound is 1-(3-(2-amino-1H-imidazol-4-yl)-2-fluorophenyl)-3-(4-(2-amino-1H-imidazol-4-yl)-3-fluorophenyl)urea (9k).

13. The composition of claim 11 wherein the antibiotic is a macrolide antibiotic.

14. The composition of claim 11 wherein the antibiotic is clarithromycin or colistin.

15. A method for potentiating an antibiotic against a bacterial infection, comprising administering an effective amount of the composition according to claim 11 to a subject having a bacterial infection, wherein the compound potentiates the antibacterial activity of the antibiotic and thereby the composition treats the subject's bacterial infection.

16. The method of claim 15 wherein the compound is 1-(3-(2-amino-1H-imidazol-4-yl)-2-fluorophenyl)-3-(4-(2-amino-1H-imidazol-4-yl)-3-fluorophenyl)urea (9k).

17. The method of claim 15 wherein the bacterial infection is caused by Enterococcus faecium, Staphylococcus aureus, Klebsiella pneumoniae, Acinetobacter baumannii, Pseudomonas aeruginosa, or an Enterobacter species.

18. The method of claim 15 wherein bacterial infection is a multi-drug resistant strain of Acinetobacter baumannii.

19. The method of claim 15 wherein the antibiotic is a Gram-negative antibiotic.

20. The method of claim 15 wherein the antibiotic is clarithromycin.
